(19) United States
(12) Patent Application Publication
(10) Pub. No.: US 2021/0383934 A1
(43) Pub. Date: Dec. 9, 2021

(54) SYSTEM, METHOD AND APPARATUS FOR REAL-TIME SCREENING, VALIDATING AND TRACKING INFECTIOUS DISEASE AT PORT OF ENTRY

(71) Applicants: Sung Oog KIM, Seoul (KR); Tung Thanh LY, Ho Chi Minh City (VN); Benjamin Tam Thanh LY, Mount Waverley (AU); Ki Uk KIM, Bucheon-si (KR)

(72) Inventors: Sung Oog KIM, Seoul (KR); Tung Thanh LY, Ho Chi Minh City (VN); Benjamin Tam Thanh LY, Mount Waverley (AU); Ki Uk KIM, Bucheon-si (KR)

(21) Appl. No.: 17/340,686

(22) Filed: Jun. 7, 2021

Related U.S. Application Data
(60) Provisional application No. 63/036,819, filed on Jun. 9, 2020.

(51) Int. Cl. G16H 50/80; G16H 10/40; G06Q 50/14; G16H 10/65; G16H 40/67; G06K 19/06; G06F 21/62; G06F 21/60; A61B 5/00

(52) U.S. Cl. CPC G16H 50/80; G16H 10/40; G06Q 50/14; G16H 10/65; G16H 40/67; G06Q 30/018; G06F 21/6245; G06F 21/602; A61B 5/6801; A61B 5/0002; G06K 19/06037

(57) ABSTRACT

The present technology is directed to real-time screening, validating, and tracking infectious diseases including Coronavirus (COVID-19) and the like at port of entry including airports, seaports, ferries, and any international border controls. The present technology can identify a personal identification code with a mobile device of a traveler upon arrival of the traveler at an international border, wherein the mobile device is equipped with wireless network connection; obtain traveler-specific information to associate with the personal identification code of the traveler; receive a diagnostic test result of the traveler to determine whether the traveler is infected; determine whether the traveler is subject to quarantine based on the traveler-specific information and the diagnostic test result; enable a location tracking system and a notification service during a quarantine period; and terminate the location tracking system and the notification service and deleting the traveler-specific information when the quarantine period is over.

### CROSS-REFERENCE TO RELATED APPLICATION

This application claims priority to U.S. Provisional Patent Application No. 63/036,819 filed on Jun. 9, 2020, the content of which is incorporated herein by reference in its entirety.

### DESCRIPTION OF THE RELATED TECHNOLOGY

The present disclosure relates to the development of a new system, method, and apparatus for real-time screening, validating, and tracking infectious diseases including Coronavirus (COVID-19) and the like at port of entry including airports, seaports, ferries, and any international border controls.

### BACKGROUND

Infectious diseases such as COVID-19 are thought to spread mainly through close contact from person-to-person, for example, between people who are in close contact with one another (e.g., within about 6 feet or 2 meters). Droplets are produced when an infected person coughs, sneezes, or talks. These droplets can land in the mouths or noses of people who are nearby or possibly be inhaled into the lungs. Some viruses, like COVID-19, may be spread by people who are not even showing symptoms.

As such, maintaining a reasonable social distance is critical in preventing the spread of such infectious diseases and viruses. Studies show that the best way to stop the spread of highly contagious viruses is through isolating infected individuals and placing individuals who might have been exposed to the virus in quarantine. People in isolation or quarantine are advised to stay at a designated location (e.g., home), separate themselves from others, monitor their health, and follow directions from their state or local health department or government authority.

### BRIEF DESCRIPTION OF THE DRAWINGS

The accompanying drawings, which are included to provide a further understanding of the disclosure and are incorporated in and constitute a part of the disclosure, illustrate aspects of the disclosure and together with the description serve to explain the principle of the disclosure.

In the drawings:

FIGS. 1A to 1K are continuous flow chart diagrams for an example method of real-time screening, validating, and tracking infectious diseases at a port of entry in accordance with one or more aspects of the present disclosure;

FIG. 2 illustrates a flow chart diagram for entry process in real-time screening, validating, and tracking of infectious diseases at a port of entry in accordance with one or more aspects of the present disclosure;

FIG. 3 illustrates a flow chart diagram for exit process in real-time screening, validating, and tracking of infectious diseases at a port of entry in accordance with one or more aspects of the present disclosure;

FIG. 4 illustrates a flow chart diagram for passport input process in real-time screening, validating, and tracking of infectious diseases at a port of entry in accordance with one or more aspects of the present disclosure;

FIGS. 5A and 5B illustrate continuous flow chart diagrams for use of a wearable device in real-time screening, validating, and tracking of infectious diseases in accordance with one or more aspects of the present disclosure;

FIGS. 6A and 6B illustrate exemplary representations of a user interface for real-time screening, validating, and tracking of infectious diseases in accordance with one or more aspects of the present disclosure; and

FIGS. 7A and 7B illustrate exemplary representations of a user interface for displaying a diagnostic test result in accordance with one or more aspects of the present disclosure.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles may be used in the examples for the convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions will control.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

In recent years, a rise in outbreaks of a deadly infectious disease, coronavirus (i.e., COVID-19) has led to an unprecedented pandemic throughout the world. This highly contagious disease can be easily transmitted from person-to-person in close proximity or even through indirect contact via objects or surfaces. Studies show that the most critical component in the prevention of the spread of the infectious disease or viruses like COVID-19, especially in the context of dense populations and frequent travels across different countries, is to take precautionary measures such as social distancing, quarantine, and isolation at a global level.

Effective control measures include official investigation, implementation of countermeasures, screening, and monitoring, for example, closure of borders or restricting inbound and outbound travelers to prevent the international spread of disease, screening travelers at a port of entry. The quarantine can effectively help prevent the spread of disease that can occur before a person knows that he is infected or when the person is infected without showing any symptoms. Also, people in isolation or quarantine are advised to stay home, separate themselves from others, monitor their health, and follow directions from their state or local health department.

Therefore, there is a need for an effective control measure and global surveillance, in particular, for real-time screening, validating, and tracking infectious diseases including Coronavirus (COVID-19) and the like at port of entry including airports, seaports, ferries, and any international border controls to ensure prevention of the spread of the infectious diseases.

The present technology includes systems, methods, and apparatus for solving these problems and discrepancies. Specifically, systems, methods, and computer-readable media for real-time screening, validating, and tracking infectious diseases including Coronavirus (COVID-19) and the like at port of entry including airports, seaports, ferries, and any international border controls. In particular, the present technology can include user registration, self-diagnosis, guidelines for self-quarantine, the transmission of location information, the transmission of the distance information, e.g., between a wearable device and a client device.

Furthermore, the present disclosure can be implemented as part of a standard operating procedure (SOP), good transportation practice (GTP), or good quarantine practice (GQP) for travelers at a border control point.

### OVERVIEW

Methods, systems, and non-transitory computer-readable media are provided for real-time screening, validating, and tracking infectious diseases including Coronavirus (COVID-19) and the like at port of entry including airports, seaports, ferries, and any international border controls. In particular, the present technology can include user registration, self-diagnosis, guidelines for self-quarantine, transmission of location information, transmission of the distance information.

The present technology can identify a personal identification code with a mobile device of a traveler upon arrival of the traveler at an international border, wherein the mobile device is equipped with wireless network connection; obtain traveler-specific information to associate with the personal identification code of the traveler; receive a diagnostic test result of the traveler to determine whether the traveler is infected; determine whether the traveler is subject to quarantine based on the traveler-specific information and the diagnostic test result; enable a location tracking system and a notification service during a quarantine period; and terminate the location tracking system and the notification service and deleting the traveler-specific information when the quarantine period is over.

The present technology can further pair with a wearable device associated with the personal identification code of the traveler. The wearable device can monitor one or more health conditions of the traveler. Also, the wearable device can monitor the movement and location of the traveler. The present technology can further store the location history of the traveler obtained from the wearable device. The traveler-specific information is obtained from an identification (ID) of the traveler or a boarding pass of the traveler. For example, the traveler-specific information is obtained from the ID of the traveler or the boarding pass of the traveler via an Optical Character Recognition (OCR) technology.

Furthermore, the present technology can record a traveler's visit on an electronic visitor directory based on the personal identification code including the traveler-specific information. Also, the present technology can receive consent from the traveler for collecting data of personal information and location information of the traveler.

The present technology can further receive information of one or more individuals and associating the information of one or more individuals with the personal identification code of the traveler. In addition, the present technology can transmit the diagnostic test result to a public health agency. The traveler is subject to quarantine if the diagnostic test result is positive. The traveler is subject to quarantine if the traveler is entering a country regardless of the diagnostic test result. However, the traveler is exempt from the quarantine if the traveler is departing a country and the diagnostic test result is negative.

Moreover, the present technology can terminate the location tracking system and the notification service and store the personal identification code when the quarantine period is over. The notification service can include transmitting a notification to a public health agency and the traveler when the traveler violates one or more guidelines of the quarantine. The personal identification code can be a QR code with encrypted personal information

A system for real-time screening, validating, and tracking infectious diseases at port of entry can include one or more processors and at least one computer-readable storage medium storing instructions which, when executed by the one or more processors, cause the one or more processors to identify a personal identification code with a mobile device of a traveler upon arrival of the traveler at an international border, wherein the mobile device is equipped with wireless network connection; obtain traveler-specific information to associate with the personal identification code of the traveler; receive a diagnostic test result of the traveler to determine whether the traveler is infected; determine whether the traveler is subject to quarantine based on the traveler-specific information and the diagnostic test result; enable a location tracking system and a notification service during a quarantine period; and terminate the location tracking system and the notification service and deleting the traveler-specific information when the quarantine period is over.

A non-transitory computer-readable storage medium having stored therein instructions which, when executed by one or more processors, can cause the one or more processors to identify a personal identification code with a mobile device of a traveler upon arrival of the traveler at an international border, wherein the mobile device is equipped with wireless network connection; obtain traveler-specific information to associate with the personal identification code of the traveler; receive a diagnostic test result of the traveler to determine whether the traveler is infected; determine whether the traveler is subject to quarantine based on the traveler-specific information and the diagnostic test result; enable a location tracking system and a notification service during a quarantine period; and terminate the location tracking system and the notification service and deleting the traveler-specific information when the quarantine period is over.

### DESCRIPTION

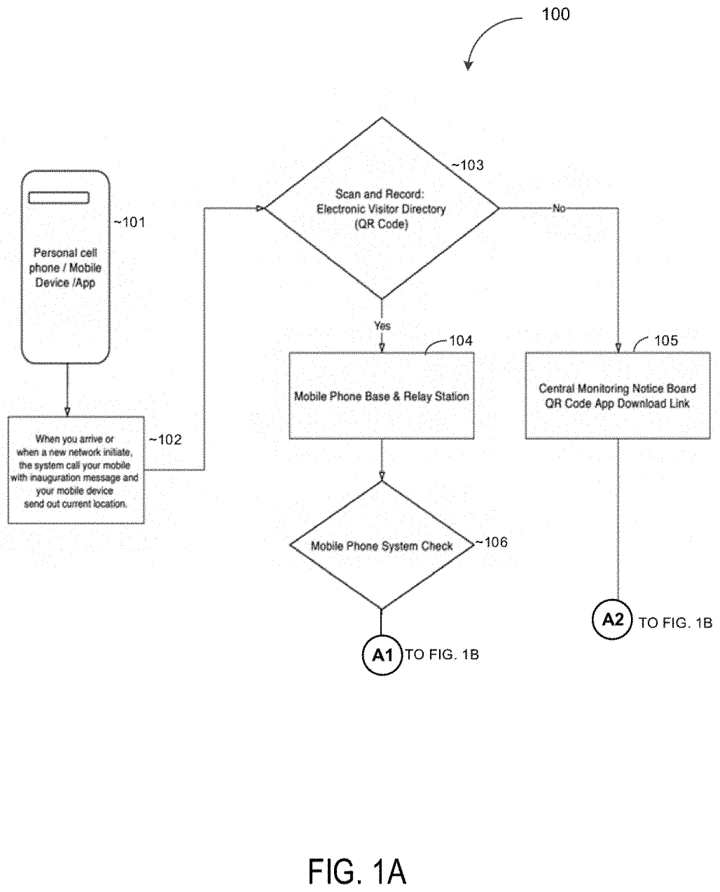

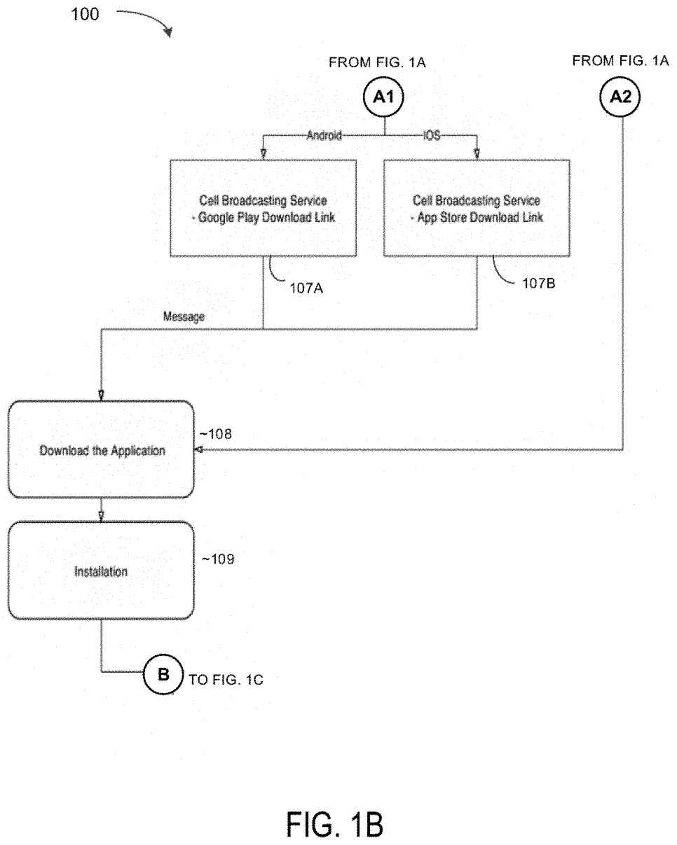

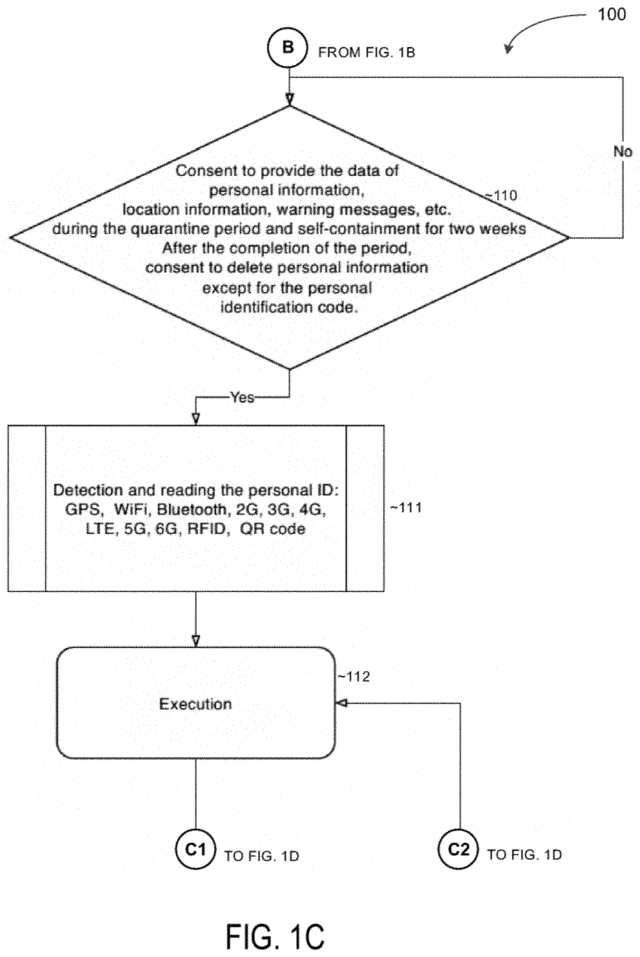

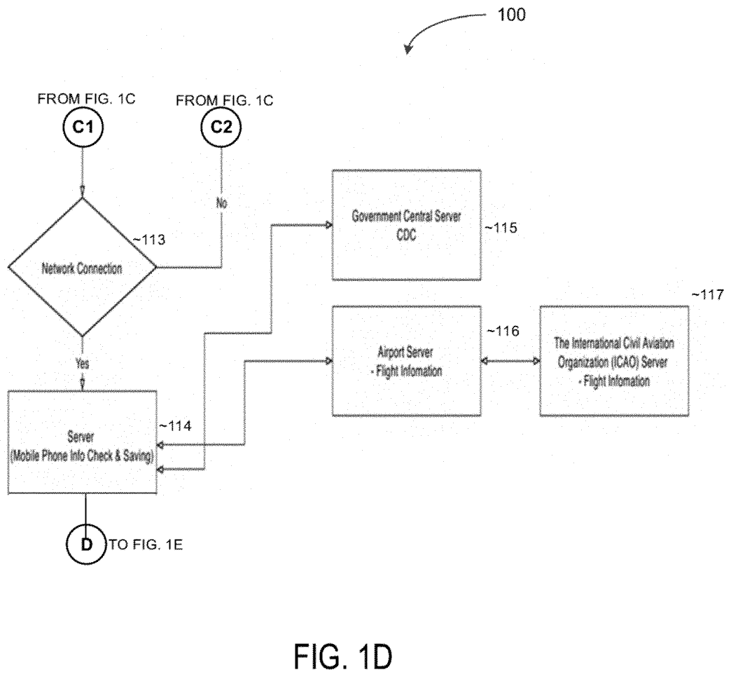

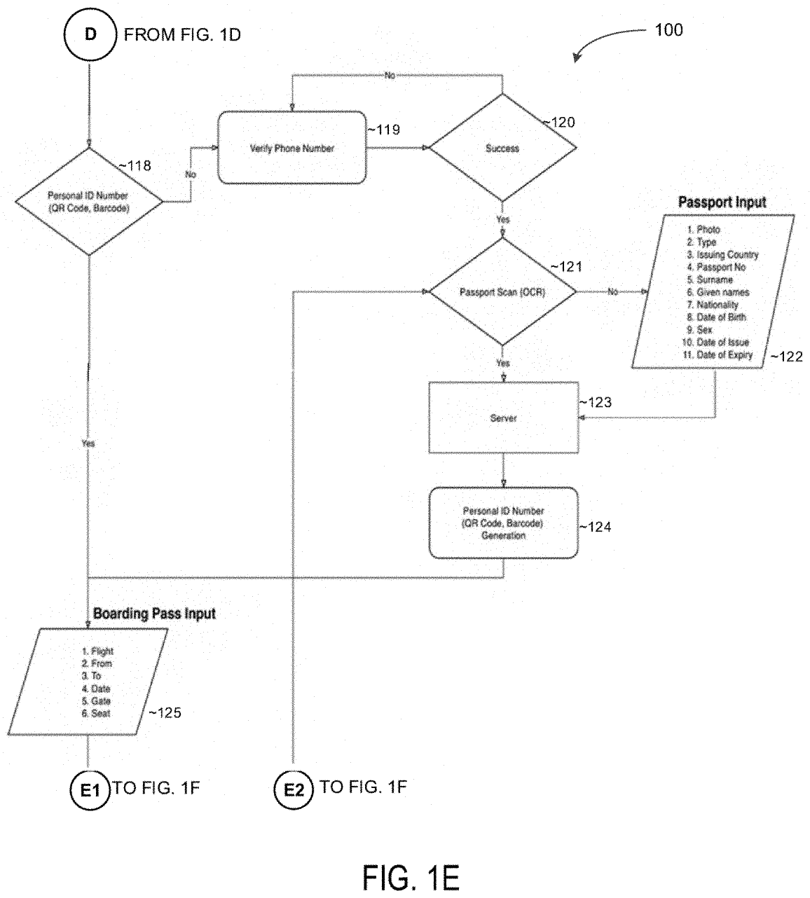

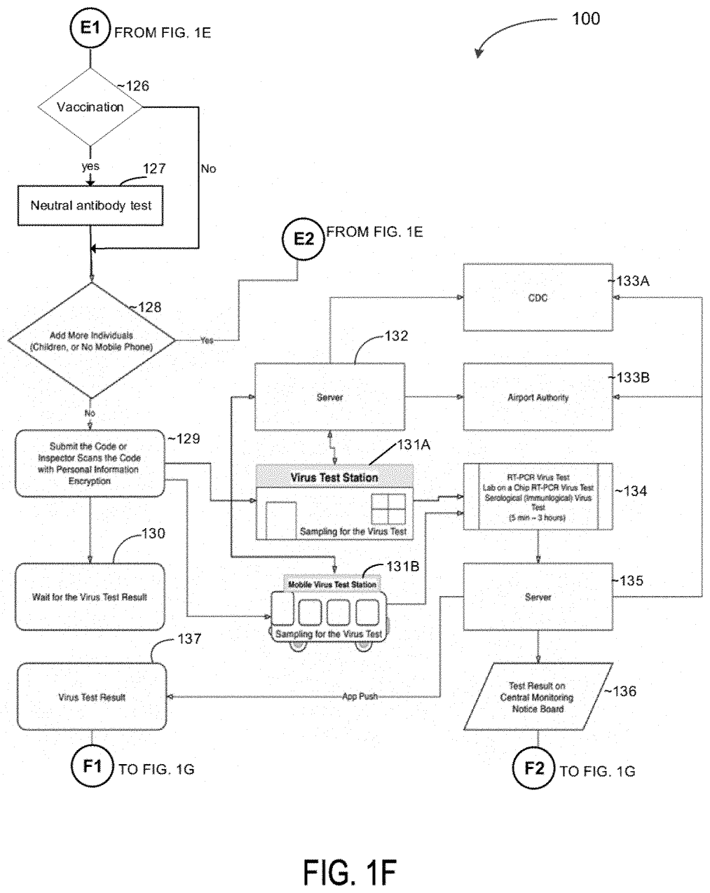

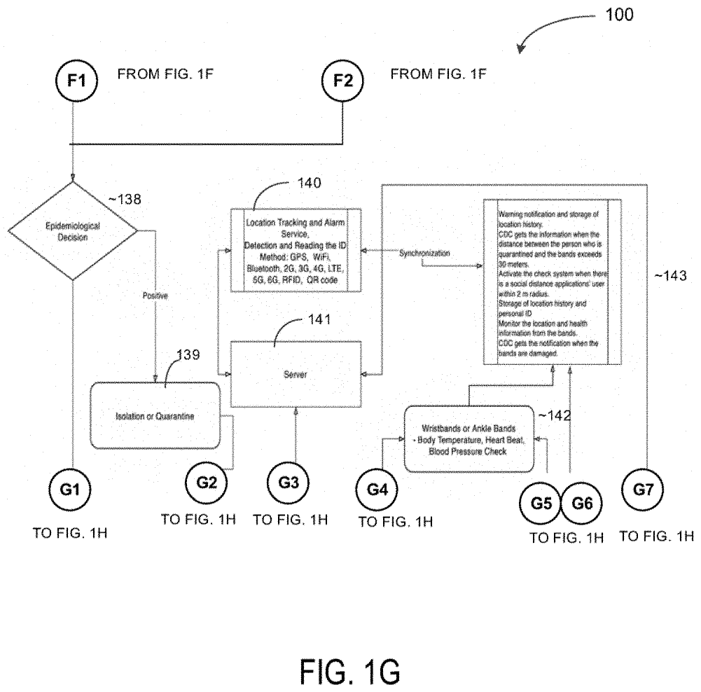

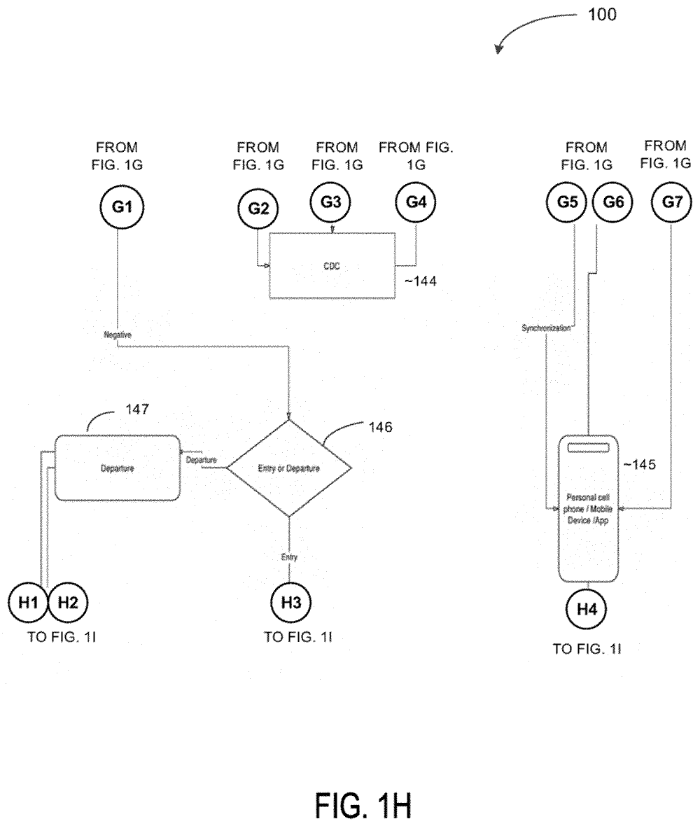

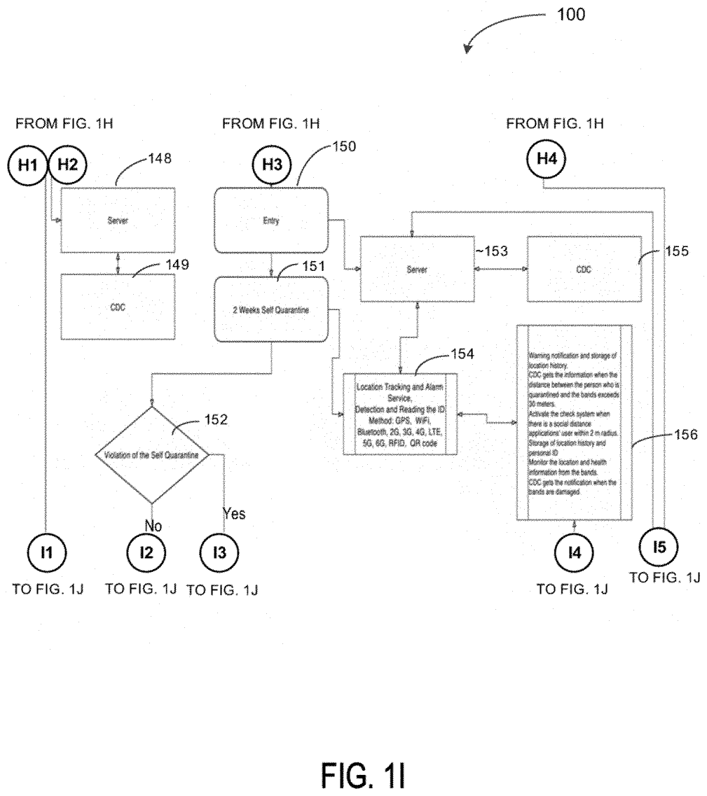

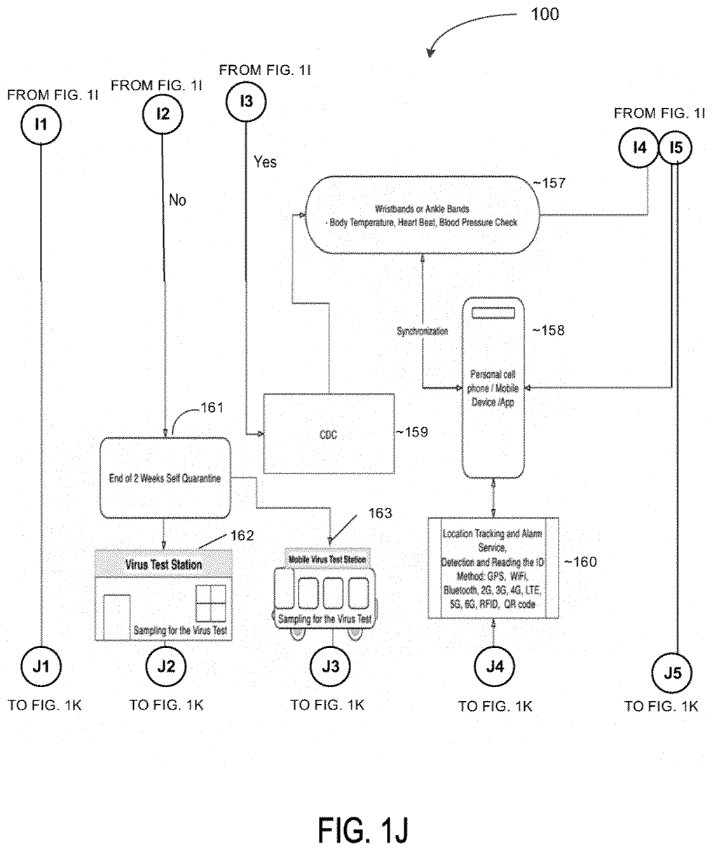

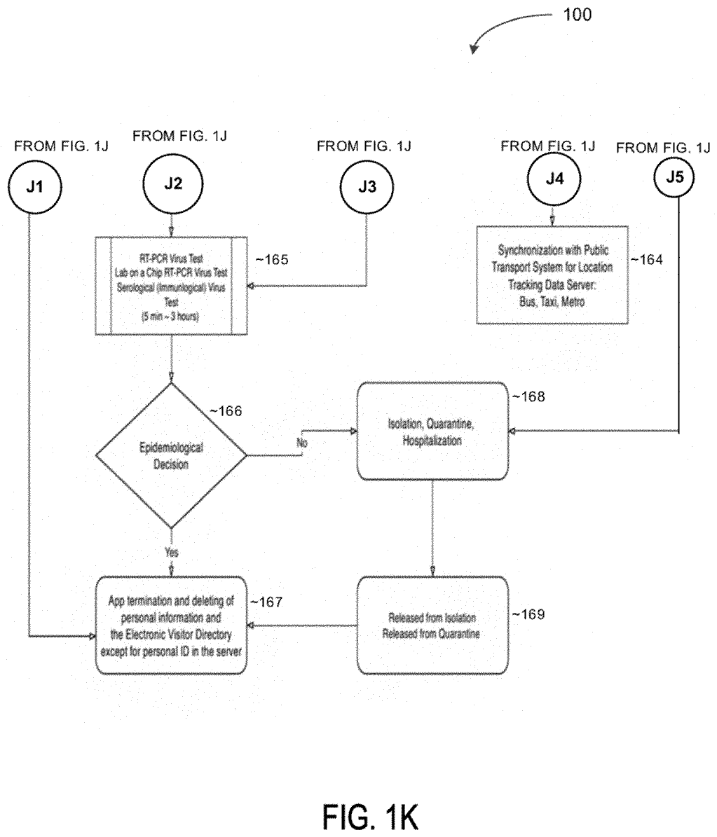

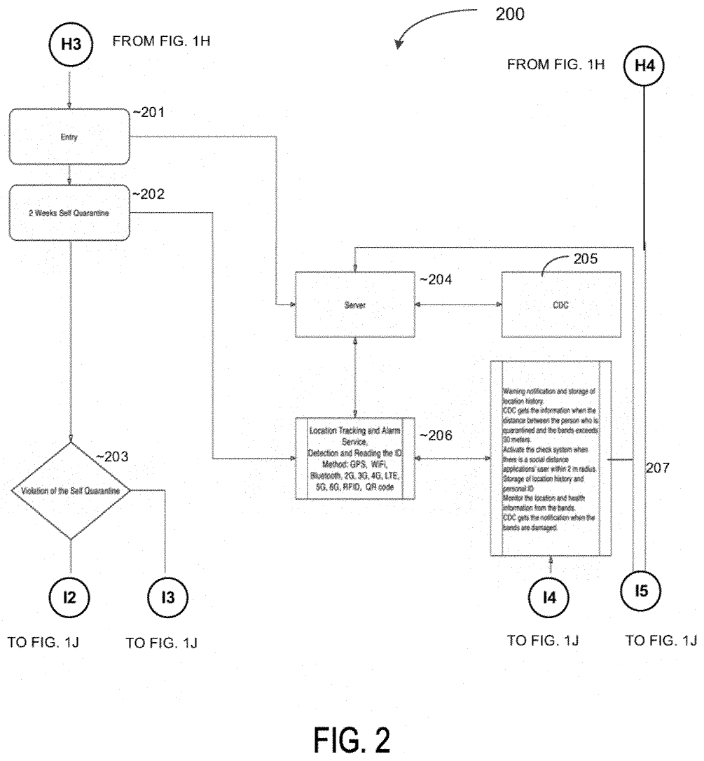

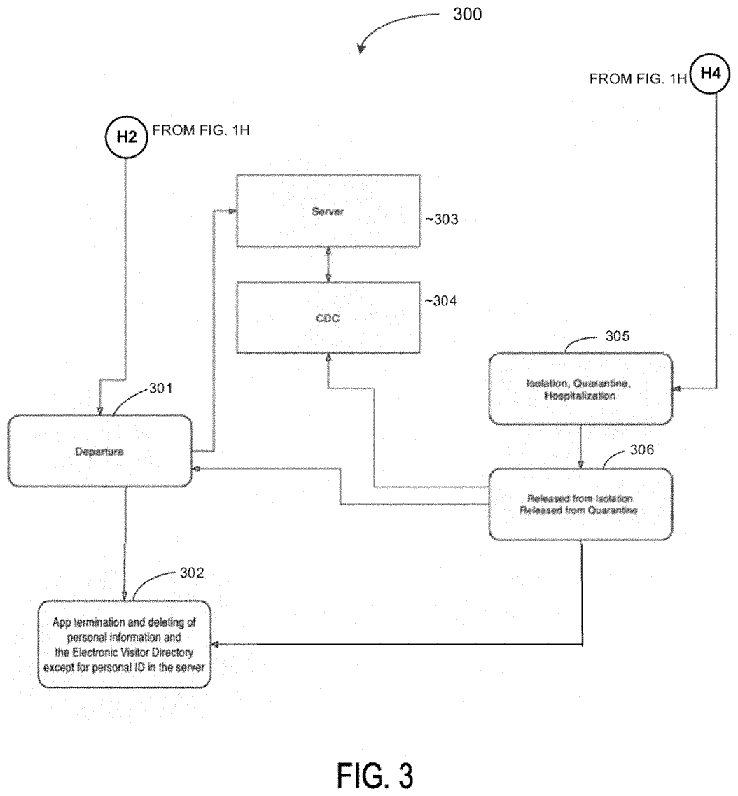

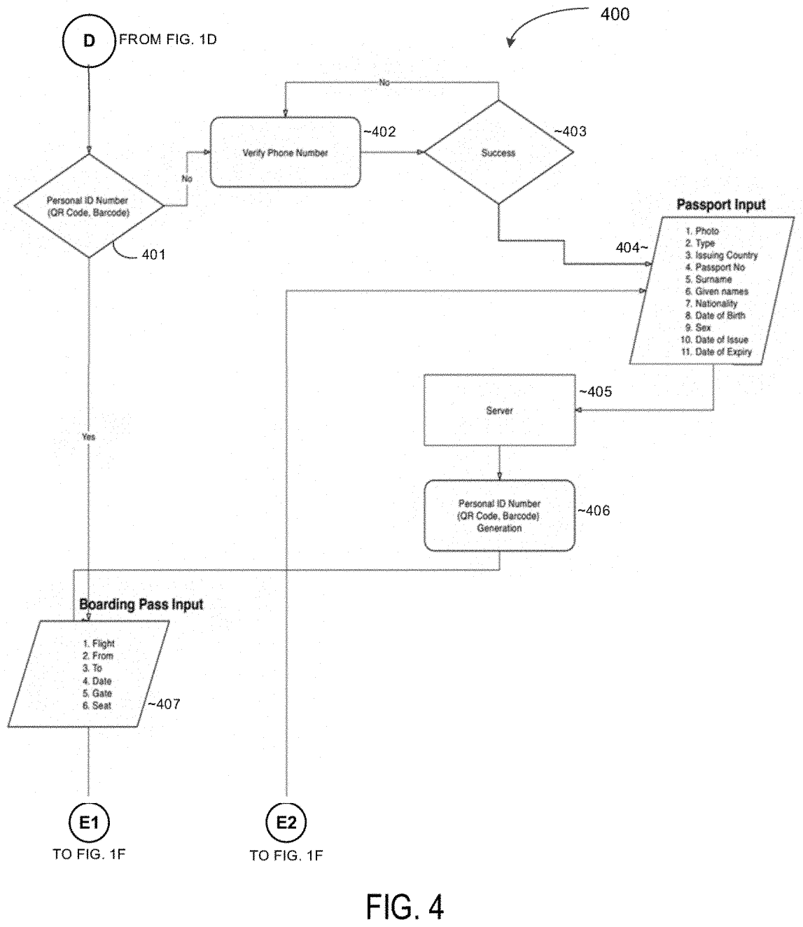

FIGS. 1A-1K illustrate continuous flow diagrams depicting an exemplary representation of a method 100 for real-time screening, validating, and tracking infectious diseases at a port of entry in accordance with one or more aspects of the present disclosure. Although example method 100 depicts a particular sequence of operations, the sequence may be altered without departing from the scope of the present disclosure. For example, some of the operations depicted may be performed in parallel or in a different sequence that does not materially affect the function of method 100. In other examples, different components of an example device or system that implements method 100 may perform functions at substantially the same time or in a specific sequence.

As depicted in FIG. 1A, in some embodiments, upon arrival at a port of entry, a public transportation terminal, or an international border control point such as airports, seaports, ferries, method 100 includes detecting a mobile device 101 (e.g., cellphone, smartphone, tablet, or smartwatch) of a traveler. Then, method 100 includes sending a welcome message to the mobile device (e.g., via a text or a call) and receiving the current location of the mobile device (step 102). In some aspects, method 100 includes receiving a unique identifier (e.g., QR code) associated with the traveler and storing the traveler's visit with the unique identifier on an electronic visitor directory (e.g., electronic entry log) (step 103). The record of the traveler's visit can be used for an accurate and rapid contact tracing (i.e., to trace any individuals who are likely exposed to the virus) in case the traveler is found to be infected with a disease or a virus. Also, the recorded information regarding the traveler's visit can be transmitted to a social security service agency or a public health agency. In some examples, method 100 includes connecting the mobile device of the traveler to mobile base stations and mobile relay stations (step 104) and determining an operating system of the mobile device, e.g., Android or iOS (step 106). If the unique identifier associated with the traveler is not available, an application download link can be connected via a central monitoring notice board where a unique identifier (e.g., QR code) can be generated (step 105).

As illustrated in FIG. 1B, method 100 includes directing the mobile device to a download link (e.g., Google Play or App Store) (steps 107A and 107B) so that an application for screening and tracking for safety and prevention of infectious diseases (e.g., self-quarantine safety protection application) can be downloaded (step 108) and installed (step 109).

As shown in FIG. 1C, in some embodiments, the traveler is provided with a consent form, i.e., the traveler is asked to consent to the collection and use of personal information including location history, and to receive a notification during the quarantine period (step 110). The collection and use of personal information (e.g., the transmission of personal information including location information and health status of the traveler) is to be in compliance with local data protection and privacy laws or any applicable local laws or regulations. The contents of the consent form can be related to the safe protection of persons in quarantine and prevention of the spread of infections. In some examples, the consent form can include a consent to delete personal information collected during the quarantine period and store only the personal identification code once the quarantine is over. The personal identification code, which is stored even after the quarantine period can be used in the future when the traveler is traveling through the same or different port of entry or border control point.

The purpose of the collection and use of any personal information is to protect infected persons in quarantine and to prevent the infectious disease or containment of the spread of disease while protecting one's privacy. For example, personal information can be kept for a certain period of time after the infectious disease is lifted (e.g., when the pandemic is over) in accordance with any applicable local data privacy and protection laws or regulations.

Once the consent is accepted, method 100 includes detecting and identifying the personal identification code with the mobile device of the traveler and verifying that the device is equipped with a wireless network connection (e.g., GPS, Wi-Fi, Bluetooth, 2G, 3G, 4G (LTE), 5G, 6G, RFID, or QR code) (step 111). Then, the application for screening, validating, and tracking infectious diseases can be opened on the mobile device (step 112).

As illustrated in FIG. 1D, method 100 includes establishing a network connection (step 113) and connecting to a server (step 114). In some instances, method 100 includes communicating with a server in the national public health agency (e.g., Centers for Disease Control and Prevention (CDC), or Ministry of Health), port of entry (e.g., airport, seaport, or ferry), or an international aviation regulator (e.g., International Civil Aviation Organization (ICAO) (steps 115-117). For example, the flight information of the traveler can be obtained based on communication with the servers in the airport or ICAO.

In some aspects, as depicted in FIG. 1E, method 100 includes identifying a personal identification code (for example, identification number, QR code, or barcode) that identifies the mobile device of the traveler (step 118). If the personal identification code is not available, verification of the traveler can be performed with a traveler's phone number for initial use (step 119). Once the verification with the phone number is successful (step 120), the traveler's personal information can be obtained based on the traveler's identification (ID) card or document (e.g., passport, driver's license, or state ID) (step 121). For example, the traveler's personal information can be obtained from the ID card (e.g., a passport) via an Optical Character Recognition (OCR) technology (i.e., data from a scanned ID card or document can be extracted via OCR). Alternatively, the traveler's personal information can be manually added (step 122). The traveler's personal information to be collected can include, for example, photo, type, issuing country, passport or identification number, surname, given name, nationality, date of birth, sex, date of issue, and date of expiry. The passport data or any personal information data, whether extracted from the scanned ID or manually added, can be sent to a server (step 123). Based on the traveler's personal information from the ID card or document data, a personal identification code can be generated (step 124). In some aspects, method 100 includes obtaining boarding pass data such as flight information, departure, destination, date, gate number, and seat number (step 125). Similar to the OCR process of the ID card or document, the boarding pass can be scanned and data therein can be automatically imported via OCR technology.

In some embodiments, as illustrated in FIG. 1F, method 100 includes asking the traveler whether the traveler has been vaccinated or not (step 126). If vaccinated, a neutral antibody test can be performed with the traveler (step 127). If not, method 100 proceeds to the next step. In some instances, only inbound travelers are subject to the vaccination question while outbound travelers are exempt from the vaccination question and further neutral antibody test. Although the example method 100 in FIG. IF depicts a particular sequence of operations, in particular with respect to steps 126 and 127, the sequence may be altered without departing from the scope of the present disclosure. For example, the vaccination question can be asked upon arrival at the border (e.g., step 102), right before taking the diagnostic test (e.g., steps 131A or 131B), or at any suitable stage of method 100. In addition, after confirmed by the neutral antibody test, a vaccinated person can bypass the diagnostic test or quarantine. Details of the exemptions from the diagnostic test or quarantine for a vaccinated person will be in compliance with local laws and regulations.

Furthermore, information about additional individuals (e.g., children or other family members of the traveler) can be added and associated with the personal identification code of the traveler (step 128). For example, children or family members that are traveling together and are subject to quarantine at the same location can be associated with a single personal identification code. Once the code is submitted or manually scanned by an inspector (step 129), a diagnostic test can be performed at a virus test station (step 131A) or mobile virus test station (step 131B). Any information related to the diagnostic test can be sent to a server (step 132), which then can be communicated with a public health agency (e.g., CDC) or a border control authority (e.g., airport authority) (steps 133A and 133B). Examples of the diagnostic test can include, but is not limited to, Reverse Transcription-Polymerase Chain Reaction (RT-PCR), lab-on-a-chip (LOC) RT-PCR test, or serological (immunological) test (step 134). In some aspects, diagnostic test results can be sent to a server (step 135), central monitoring notice board (step 136), or the user via the mobile device (e.g., via a push notification) (step 137).

As illustrated in FIG. 1G, in some examples, method 100 includes making an epidemiological decision based on the diagnostic test results (step 138), for example, to determine whether the traveler is infected with infections or viruses (i.e., a positive test result) or not (i.e., a negative test result). In a response to a positive test result, the traveler can be placed in a compulsory quarantine (step 139). Furthermore, method 100 includes enabling the tracking location of the traveler and providing a notification service (step 140). As previously described, such tracking of location and a notification service can be performed via any applicable wireless communication technology such as GPS, Wi-Fi, Bluetooth, 2G, 3G, 4G (LTE), 5G, 6G, RFID, or QR code to periodically check whether the traveler is complying with quarantine regulations. In some examples, location can be tracked based on the wireless signals from the mobile device. For example, location tracking is to ensure that the quarantined person stays in the designated location during the quarantine period for prevention of the potential spread of the infectious disease. A time period required for quarantine or rules or guidelines regarding the quarantine can be in accordance with local regulations or governed by local public health authorities.

Furthermore, data collected from the location tracking and notification service can be transmitted to a public health agency (e.g., CDC) (step 144) via a server (step 141). For example, a public official can monitor the status of self-quarantine individuals, receive results of self-diagnosis, and check the location of self-quarantine individuals. Also, a general manager can manage public officials, check the status of self-quarantine individuals, check absent without leave (AWOL) history, and review statistical data.

In some embodiments, method 100 includes pairing with a wearable device (e.g., wristband or ankle band) (step 142), which can be associated with the personal identification code of the traveler. The paring between the wearable device and the mobile device of the traveler can be performed via QR code or barcode pairing, for example, taking a picture of the QR code on the wearable device with a camera on the mobile device. Alternatively, a serial number or code of the QR code can be manually entered to connect the wearable device with the mobile device.

In some aspects, the wearable device can monitor the health conditions of the traveler, for example, body temperature, heart rate, or blood pressure that can be used to determine symptoms of the infections. When one or more of the health conditions is out of a threshold or a certain range that is likely to indicate symptoms of the infections, an automatic notification can be generated and sent to the public health agency (step 143) and the traveler via the mobile device (step 145) or the wearable device. In addition to monitoring health conditions with the wearable device, periodic self-diagnosis can be obtained to determine whether any symptoms of the infections or viruses (e.g., fever, cough, sore throat, or dyspnea) are shown.

In some instances, the wearable device can monitor the movement of the traveler or detect the location of the traveler (step 143). The location history and health information obtained from the wearable device can be stored with the traveler's personal information associated with the traveler's personal identification code (step 143). Also, any data related to the location history or health information collected by the wearable device can be accessible via the mobile device (step 145). Furthermore, the location history and health information can be shared with the public health agency (e.g., CDC) (step 144) via a server (step 141). In some examples, a check system can be activated where signals exchanged between different mobile devices can be used to detect other individuals within a certain range of distance (e.g., 2 meters). The check system can help contact tracing, i.e., identifying other individuals who have been in close contact with an infected person. In some examples, method 100 includes notifying the public health agency when any of the regulations or guidelines regarding the quarantine is in violation (for example, when the distance between the traveler and the wearable device exceeds a certain range of distance (e.g., 30 meters) or when the wearable device is damaged or malfunctioning. Furthermore, in some embodiments, the location tracking and notification service via the mobile device at step 140 can be synchronized with monitoring of the health conditions and location of the traveler via the wearable device at step 143 to ensure accurate and reliable monitoring and tracking during quarantine.

As depicted in FIG. 1H, in a response to a negative test result, method 100 includes determining the entry or departure status (step 146) of the traveler, i.e., whether the traveler is entering or departing a country at the international border. In case of a departure (e.g., outbound traveler) (step 147), a current session on the application with the mobile device can be terminated, i.e., deleting the personal information (e.g., data obtained from a passport or a boarding pass, test results) except the personal identification code and removing the traveler's information on the electronic visitor directory (step 167). In some instances, the traveler's departure information can be shared via the server (step 148) with the public health authority (step 149).

In case of an entry (i.e., inbound traveler) (step 150), as shown in FIG. 1I, the traveler can be placed into quarantine in compliance with local rules and regulations at the entering country (step 151). For example, the traveler is required to enter quarantine on the day of the entry and remain in quarantine for the next 14 days in accordance with the Quarantine Act and the Infectious Disease Control and Prevention Act in Korea. In some instances, method 100 includes enabling the tracking location of the traveler and providing a notification (step 154) via any applicable wireless communication technology (e.g., GPS, Wi-Fi, Bluetooth, 2G, 3G, 4G (LTE), 5G, 6G, RFID, or QR code). As described previously with respect to steps 140-145, similar steps can be further performed (steps 153-160). A time period required for quarantine or rules or guidelines regarding quarantine will be in compliance with local regulations or governed by local public health authorities. Furthermore, data collected from the location tracking and notification service can be transmitted to a public health agency (e.g., CDC) (step 155) via a server (step 153).

In some embodiments, method 100 includes pairing with a wearable device (e.g., wristband or ankle band) (step 157), which can be associated with the personal identification code of the traveler. As previously described, in some aspects, the wearable device can monitor the health conditions of the traveler, for example, body temperature, heart rate, or blood pressure that can be used to determine symptoms of the infections. When one or more of the health conditions is out of a threshold or a certain range that is likely to indicate symptoms of the infections, an automatic notification can be generated and sent to the public health agency (step 155) and the traveler via the mobile device (step 158) or the wearable device. In some instances, the wearable device can monitor the movement of the traveler or detect the location of the traveler (step 156). The location history and health information obtained from the wearable device can be stored as the traveler's personal information associated with the traveler's personal identification code (step 156). Also, any data related to the location history or health information collected by the wearable device can be accessible via the mobile device (step 158). Furthermore, the location history and health information can be shared with the public health agency (e.g., CDC) (step 155) via a server (step 153). In some examples, as previously described, a check system can be activated where signals exchanged between different mobile devices can be used to detect other individuals within a certain range of distance (e.g., 2 meters). The check system can help contact tracing, i.e., identifying other individuals who have been in close contact with an infected person. In some examples, method 100 includes notifying the public health agency when any of the regulations or guidelines regarding the quarantine is in violation (for example, when the distance between the traveler and the wearable device exceeds a certain range of distance (e.g., 30 meters) or when the wearable device is damaged or malfunctioning.

In some examples, method 100 includes determining whether there is a violation of quarantine during the quarantine (step 152). As depicted in FIG. 1J, when one or more rules or regulations regarding the quarantine are in violation, such violations can be reported to the public health authority (step 159). Also, the traveler in violation can be subject to periodic monitoring and tracking of health conditions and location via the wearable device (step 157), which can be synchronized with the mobile device (step 158). In some aspects, method 100 includes enabling location tracking and notification service with the mobile device of the traveler in violation (step 160). In some instances, method 100 includes synchronizing with a public transportation system (e.g., bus, taxi, or metro) to obtain more accurate and reliable location tracking data (step 164).

In some aspects, at the end of the self-quarantine (step 161), a diagnostic virus test can be performed at a virus test station (step 162) or a mobile virus test station (step 163). Examples of the diagnostic test can include, but is not limited to, Reverse Transcription-Polymerase Chain Reaction (RT-PCR), lab-on-a-chip (LOC) RT-PCR test, or serological (immunological) test (step 165).

As illustrated in FIG. 1K, based on the test results, an epidemiological decision can be made to determine whether the traveler is infected with the virus (i.e., a positive test result) or not (i.e., a negative test result) (step 166). In a response to a negative test result, a current session on the application with the mobile device can be terminated, i.e., deleting the personal information (e.g., data obtained from a passport or a boarding pass, test results) except the personal identification code and removing the traveler's information on the electronic visitor directory (step 167). On the other hand, in a response to a positive test result, the infected traveler can be placed into isolation, quarantine, or hospitalization (step 168). Once the traveler is released from isolation, quarantine, or hospitalization (step 169), a current session on the application with the mobile device can be terminated, i.e., deleting the personal information (e.g., data obtained from a passport or a boarding pass, test results) except the personal identification code and removing the traveler's information on the electronic visitor directory (step 167).

It is important to note again that the steps of method 100 illustrated in FIGS. 1A-1K represent only some of the possible examples. Some of the steps may be removed where appropriate, or modified or changed without departing from the scope of the present disclosure. In addition, a number of steps may have been described as being executed concurrently with, or in parallel to, one or more additional steps. However, the timing or sequence of these steps may be altered considerably. The flow charts described above have been presented for purposes of example and discussion.

Furthermore, the present disclosure can be implemented as part of a standard operating procedure (SOP), good transportation practice (GTP), or good quarantine practice (GQP) for travelers at the border control points. For example, a system regarding GQP for COVID-19 shall be responsible for screening and monitoring the passenger to ensure that the passenger complies with any directions issued by the authority and allow for inspection from the authority at any time without prior notice. Any information collected via the system can be made available upon request by the authority at any time. Also, the system will need to ensure that labeling is sufficient to ensure the safety and performance of the system and ensure the use of the system is by manufacturer requirements. The system shall be also responsible for establishing and implementing a method to monitor the safety and performance of the system and take the necessary actions should there be any adverse incident with regards to the use of these systems.

Additionally, the system shall maintain the distribution record and traceability. For locally manufactured ones, the manufacturers shall establish Standard Operating Procedures (SOP) on its manufacturing process and shall obtain appropriate certification within a certain time period (e.g., one-year period from the end of the emergency period). The system shall apply for registration and license the given law once the country is free from the emergency or after the expiry of the “No restriction letter.” A user or facility shall be responsible for the use of the system if the system continues to be used without registration with the authority. For the active system that requires the installation and or designated operation, the following can be provided: the manufacturer at the facility shall do the proper installation, testing and commissioning, and acceptance. Adequate on-site training by competent personnel shall be provided to the user. Information on warranty, technical support, and maintenance shall be provided to the user. The user manual shall be provided with the system. Moreover, the accessories and spare parts are available and ready to be supplied to the facilities when needed. The system shall be taken out of operations when it is no longer safe and effective for use, according to the governing law.

FIG. 2 illustrates an exemplary method 200 of entry process in real-time screening, validating, and tracking of infectious diseases at a port of entry in accordance with one or more aspects of the present disclosure. Method 200 includes determining that the traveler is entering a country (i.e., inbound traveler) (step 201). Method 200 further includes performing steps 202-207 similar to steps 151-158 as described in FIG. 1I.

FIG. 3 illustrates an exemplary method 300 of departure process in real-time screening, validating, and tracking of infectious diseases at a port of entry in accordance with one or more aspects of the present disclosure. Method 300 includes determining that the traveler is departing a country (i.e., outbound traveler) (step 301). Method 300 further includes performing steps 302-306 similar to steps 147-149 and steps 167-169 as described in FIGS. 1I and 1K.

FIG. 4 illustrates an exemplary method 400 of processing passport data and boarding pass data in accordance with one or more aspects of the present disclosure. In some embodiments, method 400 includes identifying a personal identification code (for example, identification number, QR code, or barcode) that identifies the mobile device of the traveler (step 401). If the personal identification code is not available, method 400 includes verifying the traveler with a phone number (step 402). Once the verification is successful (step 403), the traveler's personal information can be received based on the traveler's identification card (e.g., passport or driver's license) (step 404). The traveler's personal information to be collected can include, for example, photo, type, issuing country, passport or identification number, surname, given name, nationality, date of birth, sex, date of issue, and date of expiry. In some instances, the passport data can be transmitted to a server (step 405). Based on the traveler's personal information from the passport data, a personal identification code can be generated (step 406). In some aspects, method 100 includes obtaining boarding pass data such as flight information, departure, destination, date, gate number, and seat number (step 407).

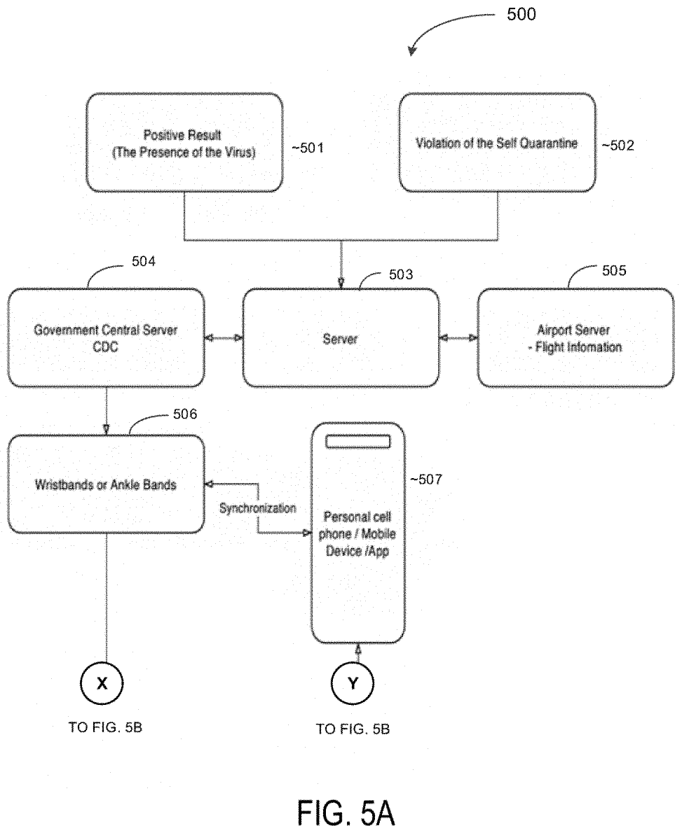

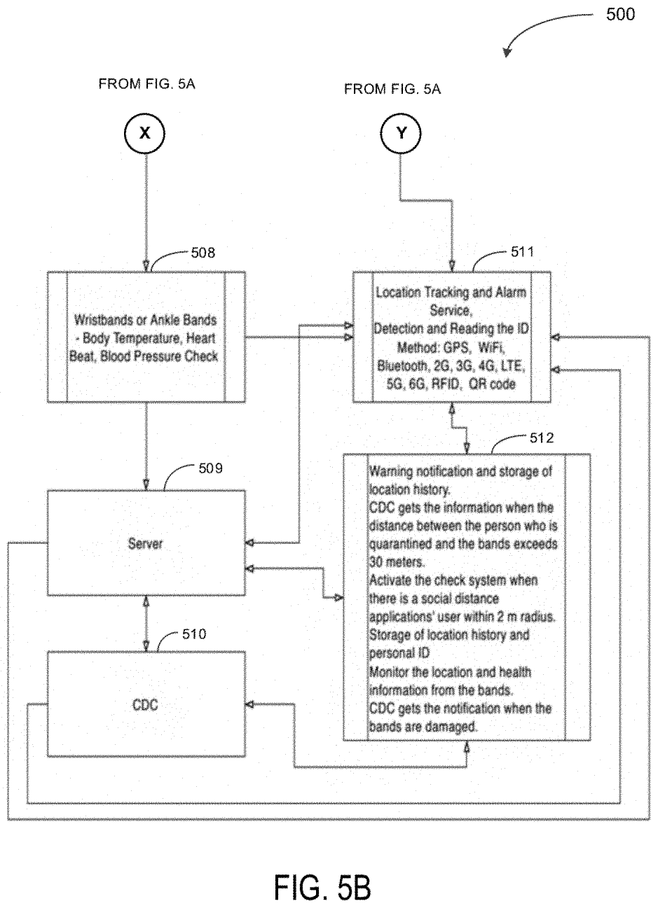

FIGS. 5A and 5B illustrate an exemplary method 500 of pairing with a wearable device in accordance with one or more aspects of the present disclosure. In a response to a positive test result (i.e., presence of the virus) (step 501) or a violation of self-quarantine (step 502), the diagnostic test result and the violation of quarantine guidelines can be reported to the public health agency (step 504) or a border control (step 505) via a server (step 503). In some embodiments, method 500 includes pairing a wearable device (e.g., wristbands or ankle bands) (step 506), which can be synchronized with the mobile device (step 507). The wearable device can monitor the health conditions of the traveler, for example, body temperature, heartrate, or blood pressure that can be used to determine symptoms of the infections (step 508). Data collected by the wearable device can be sent to a server (step 509) and shared with the public health agency or other government authority (step 510). As previously described, method 500 includes enabling location tracking and notification services (step 511). When one or more of the health conditions is out of a threshold or a certain range that is likely to indicate symptoms of the infections, an automatic notification can be generated and transmitted to the public health agency and the traveler via the mobile device (step 512) or the wearable device. In some instances, the wearable device can monitor the movement of the traveler or detect the location of the traveler (step 512). The location history and health information obtained from the wearable device can be stored as the traveler's personal information associated with the traveler's personal identification code (step 512).

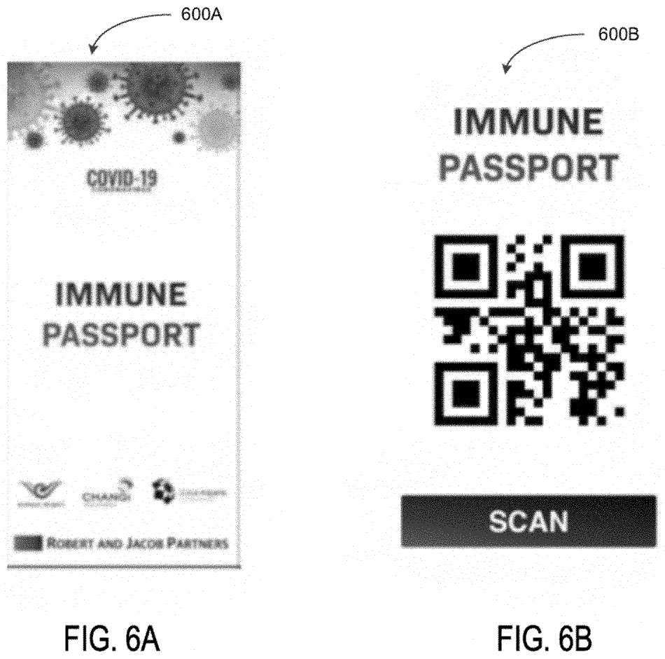

FIGS. 6A and 6B illustrate an exemplary representation of an application home screen and a personal identification code that may be accessed using a mobile device or a computing device in accordance with one or more aspects of the present disclosure. The exemplary representation of the application home screen 600A displays a home screen when the application is initiated. The exemplary representation of a personal identification code 600B displays a personal identification code, for example, a QR code, which can be scanned to identify the user.

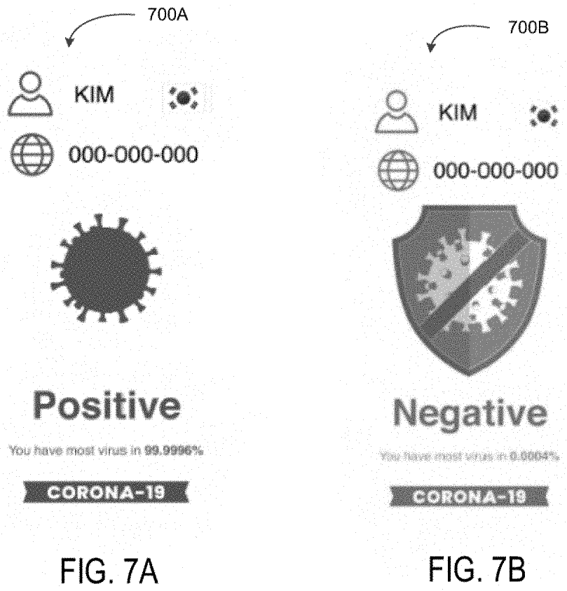

FIGS. 7A and 7B illustrate an exemplary representation of user interface 700A and 700B for displaying a diagnostic test result in accordance with one or more aspects of the present disclosure. A positive test result is shown in user interface 700A and a negative test result is shown in user interface 700B.

For clarity of explanation, in some instances, the present technology may be presented as including individual functional blocks including functional blocks comprising devices, device components, steps or routines in a method embodied in software, or combinations of hardware and software.

Any of the steps, operations, functions, or processes described herein may be performed or implemented by a combination of hardware and software services or services, alone or in combination with other devices. In some embodiments, a service can be software that resides in memory of a client device and/or one or more servers of a content management system and perform one or more functions when a processor executes the software associated with the service. In some embodiments, a service is a program, or a collection of programs that carry out a specific function. In some embodiments, a service can be considered a server. The memory can be a non-transitory computer-readable medium.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer readable media. Such instructions can comprise, for example, instructions and data which cause or otherwise configure a general purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, solid state memory devices, flash memory, USB devices provided with non-volatile memory, networked storage devices, and so on.

Devices implementing methods according to these disclosures can comprise hardware, firmware and/or software, and can take any of a variety of form factors. Typical examples of such form factors include servers, laptops, smart phones, small form factor personal computers, personal digital assistants, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims.

Claim language reciting “at least one of” refers to at least one of a set and indicates that one member of the set or multiple members of the set satisfy the claim. For example, claim language reciting “at least one of A and B” means A, B, or A and B.

What is claimed is:

1. A method for real-time screening, validating, and tracking infectious diseases at port of entry comprising:
identifying a personal identification code with a mobile device of a traveler upon arrival of the traveler at an international border, wherein the mobile device is equipped with wireless network connection;
obtaining traveler-specific information to associate with the personal identification code of the traveler;
receiving a diagnostic test result of the traveler to determine whether the traveler is infected;
determining whether the traveler is subject to quarantine based on the traveler-specific information and the diagnostic test result;
enabling a location tracking system and a notification service during a quarantine period; and
terminating the location tracking system and the notification service and deleting the traveler-specific information when the quarantine period is over.

2. The method of claim 1, further comprising:
pairing with a wearable device associated with the personal identification code of the traveler.

3. The method of claim 2, wherein the wearable device monitors one or more health conditions of the traveler.

4. The method of claim 2, wherein the wearable device monitors movement and location of the traveler.

5. The method of claim 2, further comprising:
storing location history of the traveler obtained from the wearable device.

6. The method of claim 1, wherein the traveler-specific information is obtained from an identification (ID) of the traveler or a boarding pass of the traveler.

7. The method of claim 6, wherein the traveler-specific information is obtained from the ID of the traveler or the boarding pass of the traveler via an Optical Character Recognition (OCR) technology.

8. The method of claim 1, wherein the traveler is subject to quarantine if the diagnostic test result is positive.

9. The method of claim 1, wherein the traveler is subject to quarantine if the traveler is entering a country regardless of the diagnostic test result.

10. The method of claim 1, wherein the traveler is exempt from quarantine if the traveler is departing a country and the diagnostic test result is negative.

11. The method of claim 1, further comprising:
recording a traveler's visit on an electronic visitor directory based on the personal identification code including the traveler-specific information.

12. The method of claim 1, further comprising:
receiving a consent from the traveler for collecting data of personal information and location information of the traveler.

13. The method of claim 1, further comprising:
receiving information of one or more individuals and associating the information of the one or more individuals with the personal identification code of the traveler.

14. The method of claim 1, further comprising:
transmitting the diagnostic test result to a public health agency.

15. The method of claim 1,
terminating the location tracking system and the notification service when the quarantine period is over.

16. The method of claim 1, further comprising:
storing the personal identification code when the quarantine period is over.

17. The method of claim 1, wherein the notification service includes transmitting a notification to a public health agency and the traveler when the traveler violates one or more guidelines of quarantine.

18. The method of claim 1, wherein the personal identification code is a QR code with encrypted personal information.

19. A system for real-time screening, validating, and tracking infectious diseases at port of entry comprising:
one or more processors; and
at least one computer-readable storage medium having stored therein instructions which, when executed by the one or more processors, cause the one or more processors to perform operations comprising:
identifying a personal identification code with a mobile device of a traveler upon arrival of the traveler at an international border, wherein the mobile device is equipped with wireless network connection;
obtaining traveler-specific information to associate with the personal identification code of the traveler;
receiving a diagnostic test result of the traveler to determine whether the traveler is infected;
determining whether the traveler is subject to a quarantine based on the traveler-specific information and the diagnostic test result;
enabling a location tracking system and a notification service during a quarantine period;
terminating the location tracking system and the notification service and deleting the traveler-specific information when the quarantine period is over.

20. A non-transitory computer-readable storage medium having stored therein instructions which, when executed by a processor, cause the processor to perform operations for real-time screening, validating, and tracking infectious diseases at port of entry comprising:
identifying a personal identification code with a mobile device of a traveler upon arrival of the traveler at an international border, wherein the mobile device is equipped with wireless network connection;
obtaining traveler-specific information to associate with the personal identification code of the traveler;
receiving a diagnostic test result of the traveler to determine whether the traveler is infected;
determining whether the traveler is subject to quarantine based on the traveler-specific information and the diagnostic test result;
enabling a location tracking system and a notification service during a quarantine period;
terminating the location tracking system and the notification service and deleting the traveler-specific information when the quarantine period is over.
